(19) United States
(12) Patent Application Publication
(10) Pub. No.: US 2024/0257994 A1
(43) Pub. Date: Aug. 1, 2024

(54) SIGNAL-ISOLATING CABLE FOR INTERFACING MEDICAL DEVICES

(71) Applicant: Shiratronics, Inc., Brooklyn Park, MN (US)

(72) Inventors: Scott Anthony Lambert, East Bethel, MN (US); Dennis Larson, Roseville, MN (US)

(21) Appl. No.: 18/421,131

(22) Filed: Jan. 24, 2024

Related U.S. Application Data
(60) Provisional application No. 63/442,186, filed on Jan. 31, 2023.

(51) Int. Cl. H01B 11/08; A61B 5/01; A61B 5/07; A61B 5/256; H01B 3/44; A61N 1/372

(52) U.S. Cl. CPC H01B 11/08; A61B 5/01; A61B 5/076; A61B 5/256; H01B 3/445; A61B 2562/222; A61B 2562/227; A61N 1/37229

(57) ABSTRACT

A medical system may include an external medical device, at least two patient-applied parts, and a furcated cable having one proximal end configured to be connected to the external medical device and having at least first and second distal ends configured to be connected to the patient-applied parts. The cable may include first and second types of twisted wire pairs. The second type includes wires that have a lower gauge and higher current capacity than the first type. The cable may include at least four sets of twisted wire pairs, including at least two sets of twisted wire pairs of the first type and at least two sets of twisted wire pairs of the second type. Each of the first and second distal ends include a first type and a second type of twisted wire pairs.

### PRIORITY

This application claims the benefit of priority to U.S. Provisional Patent Application Ser. No. 63/442,186, filed Jan. 31, 2023, which is incorporated by reference herein in its entirety.

### TECHNICAL FIELD

This document relates generally to medical systems or devices, and more particularly, but not by way of limitation, to signal-isolating cables such as may be used to interface with medical devices.

### BACKGROUND

Medical devices may sense signals and/or deliver a therapy. For example, medical devices may include implantable devices configured to deliver a therapy such as an electrical therapy. Implantable electrical therapy devices may include implantable neurostimulators. An example of an implantable neurostimulator includes a fully head-located implantable peripheral neurostimulation system, having one or more implantable devices, designed for the treatment of chronic head pain is a specific example of an implantable neurostimulation system. A corresponding coil assembly for each of the implantable devices may be used to interface with the implantable device. The coil assembly may be termed a patient-applied part as it contacts the patient during normal use.

A system with two or more patient-applied parts (e.g., two or more coil assemblies to interface with two or more implantable devices) may have a need to transmit more than one signal from an external medical device to each of the patient-applied parts. However, multiple signals may interfere with each other, causing “noise” in the signal. Improvements are desirable for interfacing with two or more patient-applied parts using multiple signals.

### SUMMARY

An example (e.g., “Example 1”) of a medical system includes an external medical device, at least two patient-applied parts configured to physically contact a patient during normal use, and a furcated cable having one proximal end configured to be connected to the external medical device and having at least two distal ends configured to be connected to the at least two patient-applied parts. The distal ends may include a first distal end and a second distal end. The cable may include first and second types of twisted wire pairs. The second type of twisted wire pairs include wires that have a lower gauge and higher current capacity than the first type of twisted wire pairs. The cable may include at least four sets of twisted wire pairs, including at least two sets of twisted wire pairs of the first type and at least two sets of twisted wire pairs of the second type. A first one of the at least two sets of twisted wire pairs of the first type and a first one of the at least two sets of twisted wire pairs of the second type may extend from the proximal end to the first distal end. A second one of the at least two sets of twisted wire pairs of the first type and a second one of the at least two sets of twisted wire pairs of the second type extend from the proximal end to the second distal end.

In Example 2, the subject matter of Example 1 may optionally be configured such that the external medical device includes an electrical port, and the one proximal end of the cable includes one connector plug for electrically connecting the at least four twisted wire pairs in the cable to the electrical port.

In Example 3, the subject matter of any one or more of Examples 1-2 may optionally be configured such that the first one of the at least two sets of twisted wire pairs of the first type and the first one of the at least two sets of twisted wire pairs of the second type are hardwired at the first distal end to a first one of the at least two patient-applied parts, and the second one of the at least two sets of twisted wire pairs of the first type and the second one of the at least two sets of twisted wire pairs of the second type are hardwired at the second distal end to a second one of the at least two patient-applied parts.

In Example 4, the subject matter of any one or more of Examples 1-3 may optionally be configured such that each of the at least four sets of twisted pairs has a conductive shield.

In Example 5, the subject matter of Example 4 may optionally be configured such that the external medical device includes a reference or ground node, and the conductive shields are terminated together and electrically connected to the reference or ground node in the external medical device.

In Example 6, the subject matter of any one or more of Examples 4-5 may optionally be configured such that the conductive shield includes a tinned copper spiral wound shield.

In Example 7, the subject matter of Example 6 may optionally be configured such that each wire within the at least four sets of twisted wire pairs is insulated with Perfluoroalkoxy (PFA) and each of the conductive shields are insulated with PTFE tape.

In Example 8, the subject matter of any one or more of Examples 1-7 may optionally be configured such that the furcated cable is a zip cord style cable configured to be pulled apart into at least a first distal segment near the first distal end and a second distal segment near the second distal end, the first distal segment includes distal portions of the first one of the at least two sets of twisted wire pairs of the first type and the first one of the at least two sets of twisted wire pairs of the second type, and the second distal segment includes distal portions of the second one of the at least two sets of twisted wire pairs of the first type and the second one of the at least two sets of twisted wire pairs of the second type.

In Example 9, the subject matter of Example 8 may optionally be configured such that the cable includes a yoke to keep the zip cord style cable from separating into the first and second distal segments past the yoke.

In Example 10, the subject matter of any one or more of Examples 8-9 may optionally be configured such that the zip cord style cable includes a first insulator jacket around the first one of the at least two sets of twisted wire pairs of the first type and the first one of the at least two sets of twisted wire pairs of the second type, and a second insulator jacket around the second one of the at least two sets of twisted wire pairs of the first type and the second one of the at least two sets of twisted wire pairs of the second type. The first insulator jacket may be adjacent and fused to the second insulator jacket. The first and second insulator jackets may be configured to be separated along the fuse to provide the first distal segment using the first insulator jacket and provide the second distal segment using the second insulator jacket.

In Example 11, the subject matter of Example 10 may optionally be configured such that the first and second insulator jackets include polyvinyl chloride (PVC).

In Example 12, the subject matter of any one or more of Examples 10-11 may optionally be configured to further include a first conductive jacket shield around the first one of the at least two sets of twisted wire pairs of the first type and the first one of the at least two sets of twisted wire pairs of the second type, and a second conductive jacket shield around the second one of the at least two sets of twisted wire pairs of the first type and the second one of the at least two sets of twisted wire pairs of the second type. The first insulator jacket may coat the first conductive jacket shield, and the second insulator jacket may coat the second conductive jacket shield.

In Example 13, the subject matter of any one or more of Examples 10-11 may optionally be configured such that the patient-applied part includes a coil assembly configured for use to be placed on the patient and used to charge, program, or communicate with an implantable device; or at least one electrode configured to contact the patient, wherein the at least one electrode is used to deliver an electrical therapy to the patient or to sense an electrical signal from the patient.

In Example 14, the subject matter of any one or more of Examples 1-13 may optionally be configured such that the first type and the second type of twisted wire pairs include 32 AWG and 30 AWG wires, respectively.

In Example 15, the subject matter of any one or more of Examples 1-14 may optionally be configured such that the medical system is configured to diagnose, treat or monitor a patient or to compensate or alleviate a disease, an injury or a disability experienced by the patient by the at least two patient-applied parts physically contacting the patient during normal use.

An example (e.g., “Example 16”) of a medical system for communicating with at least two implanted devices includes at least two coil assemblies corresponding to the at least two implanted medical devices, an external medical device, and a cable. Each of the at least two coil assemblies is configured for use to charge, program, and/or communicate with a corresponding one of the at least two implanted devices. The cable provides an electrical connection between the external medical device and the at least two coil assemblies. The cable includes first and second types of twisted wire pairs. The second type of twisted wire pairs are configured to conduct higher power signals than the first type of twisted wire pairs. The cable includes at least four sets of twisted wire pairs, including at least two sets of twisted wire pairs of the first type and at least two sets of twisted wire pairs of the second type. Each of the at least two coil assemblies is connected to one of the at least two sets of twisted wire pairs of the first type and is further connected to one of the at least two sets of twisted wire pairs of the second type.

In Example 17, the subject matter of Example 16 may optionally be configured such that the first type and the second type of twisted wire pairs include 32 AWG and 30 AWG wires, respectively.

In Example 18, the subject matter of any one or more of Examples 16-17 may optionally be configured to further include a headset. The at least two coil assemblies include a first coil assembly attached to the headset and a second coil assembly attached to the headset.

In Example 19, the subject matter of any one or more of Examples 16-18 may optionally be configured such that the second type of twisted wire pairs includes larger gauge wires than the first type of twisted wire pairs. The second type maybe used to provide at least one of: power or recharging to the implanted device, communication with the implanted device, or programming signals to program the implanted devices

In Example 20, the subject matter of any one or more of Examples 16-19 may optionally be configured such that the first type of twisted wire pairs includes smaller gauge wires than the second type of twisted wire pairs. The first type may be used to provide sensor signals.

In Example 21, the subject matter of Example 20 may optionally be configured such that the sensor signals include a temperature sensor signal.

An example (e.g., “Example 22”) of a medical system may include a headset, an external medical device, and a cable. The headset may include a first coil assembly and a second coil assembly. The headset may be configured to be worn on the head to place the first coil assembly against the head over a first head-located implant and place the second coil assembly against the head over a second head-located implant. The external medical device may include an electrical port. The cable provides an electrical connection between the external medical device and the first and second coil assemblies. The external medical device may be configured to charge, program, and/or communicate with the first and second head-located implants using the first and second coils assemblies, respectively. The cable may include a proximal end and a bifurcated distal end forming a first distal segment with a first distal end and forming a second distal segment with a second distal end. The proximal end may include a single connector plug for connecting to the electrical port. The cable may include four twisted wire pairs. The single connector plug is configured to electrically connect the four twisted wire pairs to the external medical device via the electrical port. The four twisted wire pairs include first and second twisted wire pairs of a first gauge and first and second twisted wire pairs of a second gauge larger than the first gauge. Each wire within the at least four twisted wire pairs is coated with Perfluoroalkoxy (PFA) and a first conductive shield around the first twisted wire pair and a second conductive shield around the second twisted wire pair are insulated with PTFE tape. The cable may include a first insulator jacket around the first conductive shield around the first twisted pair of the first gauge and the first twisted pair of the second gauge, and a second insulator jacket around the second conductive shield around the second twisted pair of the first gauge and the second twisted pair of the second gauge. The first insulator jacket may be fused with the second insulator jacket such that the cable is a zip cord style cable capable of having the first and second insulator jackets separated to form a first distal segment and a second distal segment. The first twisted pair of the first gauge and the first twisted pair of the second gauge in the first distal segment may be hardwired to the first coil assembly, and the second twisted pair of the first gauge and the second twisted pair of the second gauge may be hardwired to the second coil assembly.

In Example 23, the subject matter of Example 22 may optionally be configured such that the zip cord style cable includes a yoke configured to limit separation of the first and second insulator jackets into the first and second distal segments, respectively.

In Example 24, the subject matter of any one or more of Examples 22-23 may optionally be configured such that each of the at least four sets of twisted pairs has a conductive shield.

In Example 25, the subject matter of Example 24 may optionally be configured such that the external medical device includes a reference or ground node, and the conductive shields are terminated together and electrically connected to the reference or ground node in the external medical device.

In Example 26, the subject matter of any one or more of Examples 24-25 may optionally be configured to further include a first conductive jacket shield corresponding to the first insulator jacket and a second conductive jacket shield corresponding to the second insulator jacket. The first conductive jacket shield may be wrapped around the shield of the first twisted pair of the first gauge and the first twisted pair of the second gauge. The second conductive jacket shield may be wrapped around the shield of the second twisted pair of the first gauge and the second twisted pair of the second gauge.

In Example 27, the subject matter of Example 26 may optionally be configured such that the first and second conductive jacket shields are insulated with polytetrafluoroethylene (PTFE) tape.

In Example 28, the subject matter of any one or more of Examples 22-27 may optionally be configured such that the first gauge is 32 AWG and the second gauge is 30 AWG.

In Example 29, the subject matter of any one or more of Examples 22-28 may optionally be configured such that the headset further includes a sensor. The external medical device may be configured to use the first and second twisted pairs of the first gauge to communicate with the sensor, and may be configured to use the first and second twisted pairs of the second gauge to charge, program, and communicate with the head-located implants.

In Example 30, the subject matter of any one or more of Examples 22-29 may optionally be configured such that the cable further includes shielding, the electrical port includes a multi-pin connector, and the connector plug is configured to be inserted into the multi-pin connector. A plurality of pins may be used to connect to the four twisted wire pairs, and at least one pin is used to connect shielding to a reference node or ground node within the external medical device.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an implantable medical device implanted beneath the skin and over a patient's cranium.

FIG. 2 illustrates, by way of example and not limitation, an external medical device and headset configured for use to communicate with and/or charge the implantable medical device(s).

FIG. 3 illustrates, by way of example and not limitation, a side view of fully head-located neurostimulation system.

FIG. 4 illustrates, by way of example and not limitation, a block diagram of a medical device system that includes a signal isolating cable.

FIG. 5 illustrates, by way of example and not limitation, a plug or connector on a proximal end of the cable for connection to the port of the external medical device.

FIG. 6 illustrates, by way of example and not limitation, a port in the external medical device for connecting to the plug on the proximal end of the cable (see FIG. 5).

FIG. 7 illustrates, by way of example and not limitation, a schematic diagram for the cable.

FIG. 8 illustrates, by way of example and not limitation, a cross-section of the cable with a zip cord construction.

FIG. 9 illustrates a block diagram of the cable with the zip cord construction with a proximal end for connecting to the external medical device and with separated distal segments for connecting to the two coil assemblies.

FIG. 10 illustrates a headset with wires within the distal ends of the distal segments hardwired to coil assemblies on the headset, and illustrates an example of a yoke

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments of the present subject matter provide an improved cable construction for isolating multiple signals transmitted and received from at least two patient-applied parts. This cable construction provides for a single cable that connects two or more patient-applied parts that may provide an interface to two or more implantable devices. The single cable is less cumbersome, easier to manage, and reduces system cost compared to using two or more cables for such a connection. The cable size, weight, and cost may be reduced by sizing the conductors based on the signal power requirements, while maintaining signal isolation. For example, some embodiments isolate multiple higher and lower power signals within a single cable to connect to multiple patient-applied parts. The isolation addresses a potential interference problem when multiple signals are combined within a single cable. Such interference may prevent the system, such as a medical system with medical device(s), from performing intended functions.

Some embodiments of a cable include two sets of higher-power-signal and lower-power-signal wires used to connect to two patient-applied parts to interface with two implantable devices. A patient-applied part physically contacts the patient during normal use of the device. Examples of patient-applied parts include, but are not limited to, electrical contacts (e.g., electrodes) used to deliver an electrical therapy. For example, a transcutaneous electrical nerve stimulation (TENS) system may use electrodes to deliver the TENS therapy. Patient-applied parts may also include electrical contacts to sense electrical signals in the patient, or other sensors. Examples of other sensors include, but are not limited to, temperature sensors, galvanic skin response sensors, pulse oximeters, blood pressure sensors, and the like. By way of example and not limitation, the patient-applied part may include a coil assembly configured to be positioned in contact with the patient and over a medical device implant and used to communicate, program and/or recharge the medical device implant. The wires may include twisted wire pairs. Each wire within a twisted pair is an insulated wire. Each twisted wire pair includes a signal conductor and signal return conductor. Twisting the signal and return conductor wire pairs reduces incoming and outgoing noise by reducing the electric and magnetic fields as the signal and return conductor fields are of opposite polarity and cancel part of each other's fields that can interfere with the other twisted wire pairs.

The cable may include sets of twisted wire pairs, separated into two sets of two twisted wire pairs. Each set of two twisted wire pairs includes one higher-power-signal twisted pair and one lower-power-signal twisted pair. The higher-power-signal twisted pair is larger and has more current capacity than the lower-power-signal twisted pair. Each set of higher-power-signal and lower-power-signal twisted wire pairs may be connected on a distal end to one of the patient-applied parts (e.g., one of the coil assemblies). The four twisted wire pairs may be connected on the proximal end to a connector or plug used to connect to an external medical device (e.g., charger) that may produce and process the higher-power signals on the higher-power-signal twisted pairs and may process the lower-power signals on the lower-power-signal twisted pairs. Higher power signals on the higher-power-signal twisted pairs may be used for power, recharging, communications, and programming signals, and may be carried on lower gauge, higher current capacity wires. Lower power signals on the lower-power-signal twisted pairs may be used for patient monitoring signals and may be carried on higher gauge, lower current capacity wires.

Each twisted wire pair may be shielded with a conductive shield wrapped around the twisted wire pair to reduce the interference on other twisted wire pairs. In addition to shielding each twisted wire pair, each set of higher-power-signal and lower-power-signal twisted wire pairs may also be shielded to reduce the interference from one set of higher and lower power signals for one patient-applied-part to another set of higher and lower power signals for another patient-applied-part. All the shields may be terminated together at the connector to the external medical device and may connect to the system ground via the external medical device so that the system is less affected by any common mode interference from the signals and shields.

The cable may have a zip cord style cable construction to further isolate one set of higher-power-signal and lower-power-signal twisted wire pairs for one patient-applied-part from another set of higher-power-signal and lower-power-signal twisted wire pairs for another patient-applied-part. The zip cord style construction provides an insulating jacket between the sets of higher-power-signal and lower-power-signal twisted wire pairs, which provides a physical separation between the sets and prevents the shields from contacting each other. This separation reduces the interference from one set of higher and lower power signals to another. The insulating jackets within the zip cord style construction may be easily separated by pulling apart one set of shielded higher and lower power signals from the other. This allows the cable to be separated into separate distal end segments for connecting patient-applied parts at different locations. A yoke or O-ring component may keep the zip cord style cable from further separation at a preferred location on the zip cord.

The signal-isolating cable may be implemented in a medical system with at least one medical device. By way of example and not limitation, a medical device system may include implantable medical device(s) and an external medical device configured for use to communicate with, program, and/or charge the implantable medical device(s). More particularly, the system may include a fully head-located neurostimulator(s) designed for the treatment of chronic head pain. The system may be configured to provide neurostimulation therapy for chronic head pain, including chronic head pain caused by migraine and other headaches, as well as chronic head pain due other etiologies. For example, the system may be used to treat chronic head and/or face pain of multiple etiologies, including migraine headaches; and other primary headaches, including cluster headaches, hemicrania continua headaches, tension type headaches, chronic daily headaches, transformed migraine headaches; further including secondary headaches, such as cervicogenic headaches and other secondary musculoskeletal headaches; including neuropathic head and/or face pain, nociceptive head and/or face pain, and/or sympathetic related head and/or face pain; including greater occipital neuralgia, as well as the other various occipital neuralgias, supraorbital neuralgia, auriculotemporal neuralgia, infraorbital neuralgia, and other trigeminal neuralgias, and other head and face neuralgias. The use of the cable with in such a medical system is discussed here. However, the cable may be used with other medical systems.

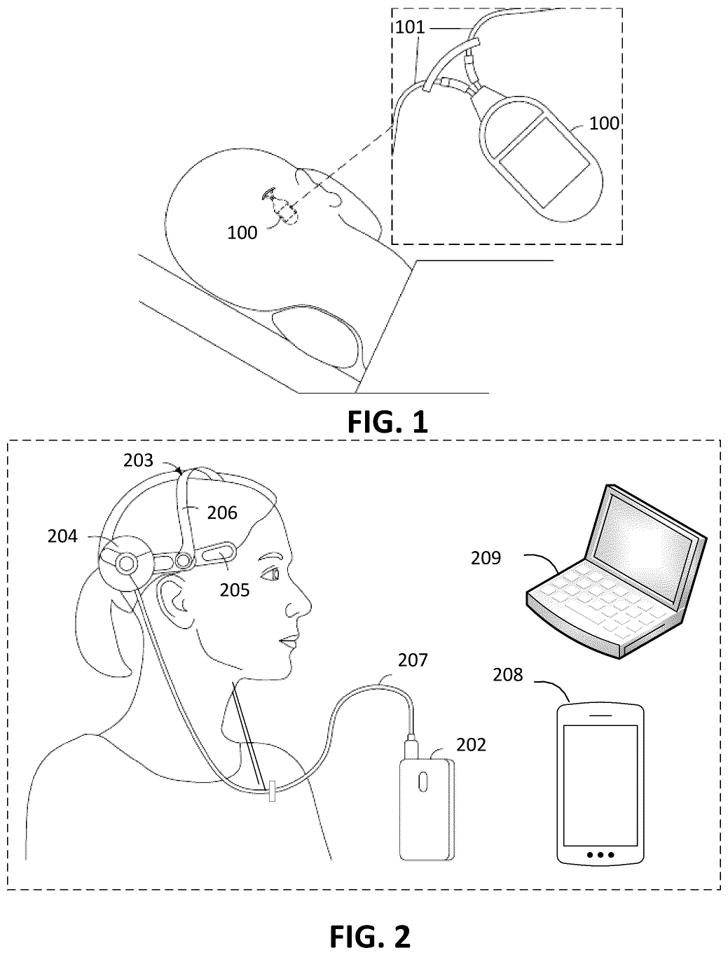

FIG. 1 illustrates, by way of example and not limitation, an implantable medical device 100 implanted beneath the skin and over a patient's cranium. The device may be referred to as a fully-head-located implantable device. The device 100 is illustrated as being implanted behind and above the ear. The implantable medical device 100 may include one or more leads 101 that may be subcutaneously tunneled to a desired neural target. The lead(s) may be integral to the device 100, as they have proximal lead ends that extend into housing of the device where electrical connections are made between stimulator circuitry and electrodes on the lead. When the medical device has integral leads, the leads are not removably connected to the device. The number of electrodes and spacing may be such as to provide therapeutic stimulation over any one or any combination of the supraorbital, parietal, and occipital region substantially simultaneously. The implantable medical device 100 may be configured to independently control each electrode to determine whether the electrode will be inactive or configured as a cathode or an anode. One or more electrodes on the lead(s) may be configured to function as an anode, and one or more electrodes on the lead may be configured to function as a cathode. For example, bipolar neuromodulation may be delivered using one or more anodes and one or more cathodes on the lead(s). A clinician may program the electrode configurations to provide a neuromodulation field that captures a desired neural target for the therapy.

FIG. 2 illustrates, by way of example and not limitation, an external medical device 202 and headset 203 configured for use to communicate with and/or charge the implantable medical device(s). The headset 203 may include an external coil 204 used to provide the communication/programmimg/charging functions, and the headset 203 may be configured to position the external coil over an implantable medical device. The headset 203 may include two external coils to be positioned over two implantable medical devices, where one of the implantable medical devices is on each side of the head. The external coils may be considered to be a patient-applied device as it is in contact with the patient during normal use. The headset 203 may include, for example, an adjustable frame 205 on each side of the head that can rotate about a point on a main headset frame 206, and may be configured to provide additional degrees of motion (e.g., sliding or pivoting motion) with respect to the main headset frame 206. These adjustable frames may be used to position the external coils 204 over the implantable medical devices when the main headset frame 206 is worn. The external medical device 202 may be electrically connected to the external coil 204 via a cable 207. In some embodiments, the external medical device 202 may be wirelessly connected to the headset 203. The external medical device 202 may communicate, using wireless or wired communication technology, with other external medical devices such as a phone 208 or programmer 209.

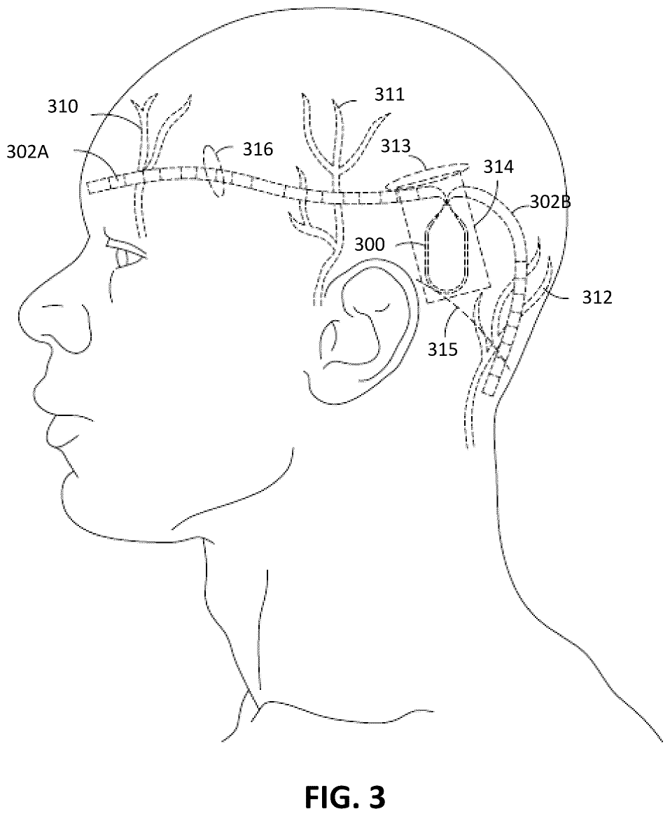

FIG. 3 illustrates, by way of example and not limitation, a side view of fully head-located neurostimulation system. Also illustrated are the supraorbital nerve 310, the auriculotemporal nerve 311 and the occipital nerve 312. The illustrated system includes a medical device (e.g., pulse generator) 300 and both a first lead 302A and a second lead 302B attached to the medical device 300. The medical device 300 and leads 302A and 302B are configured for use in stimulating the supraorbital nerve 310 toward the front of the head and the occipital nerve 312 toward the back of the head.

The patient may have had a period of trial neurostimulation, which is standard in traditional neurostimulator evaluations but is optional here. The actual permanent implant may occur in a standard operating suite with appropriate sterile precautions. By way of example and not limitation, the patient may be prepped and draped. The patient may be administered prophylactic antibiotics, local anesthetic, and sedation. The patient may be placed in a supine position with a head of the bed elevated to approximately thirty degrees. The patient's head may be turned to better access the intended implant location. While the implantable medical device may be positioned subcutaneously anywhere, it may be positioned above and behind the ear in this illustrated embodiment. Thus, a first incision 313 of sufficient length (approximately 4-6 cm) is made to a depth sufficient to reach the subcutaneous layer. A pocket 314 to accept the medical device 300 is fashioned by standard dissection techniques. The pocket 314 may be directed below the incision. The pocket 314 may be angled depending on the desired orientation of the medical device. For example, the pocket 314 may be angled posteriorly, as illustrated. The pocket 314 may be 10-20% larger than the medical device 300 to allow for a comfortable fit and no undue tension on the overlying skin and/or incision. The first incision 313 may be made and the pocket 314 formed so that the implantable medical device abuts against the nuchal ridge 315 when fully inserted into the pocket 314. The first incision 313 should not interfere with the implanted medical device 300. The present subject matter may use template(s) to help make the incision in a desired location.

A second incision 316 may be made to the subcutaneous layer at a point above and anterior to the pinna of the ear in the temple region to assist with subcutaneously routing the first lead 302A. The first lead 302A may be passed from the medical device 300 in the pocket 314 to the second incision 316, and then passed from the second incision 316 to its final subcutaneous position over supraorbital nerves 310. The second lead 302B may be passed from the medical device 300 in the pocket 314 back toward the occipital nerve 312. The medical device 300 may be inserted into the pocket 314 either before or after the leads 302A and/or 302B are tunneled to their final subcutaneous position to deliver therapy.

Tubular introducer(s) with a plastic-peel away shell may be used to assist with lead placement. However, other techniques may be used to subcutaneously tunnel the leads to their final placement to deliver the neurostimulation therapy. Following the entire placement of the complete system, including the medical device and both leads and suturing, the medical device may be powered-up and its circuits checked. Upon recovery from anesthesia the system may be turned on for the patient with a portable programmer and the multiple parameters for the system may be programmed to provide a desired therapy for the patient.

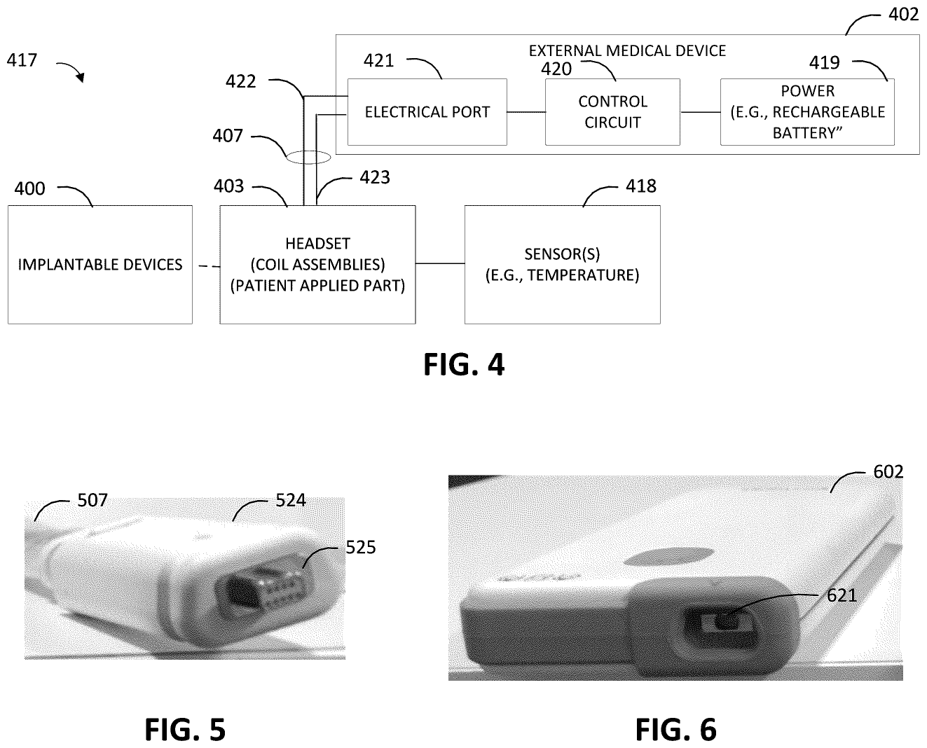

FIG. 4 illustrates, by way of example and not limitation, a block diagram of a medical device system 417 that includes a signal isolating cable 407. The medical device system 417 may include an external medical device 402 connected via a headset cable 407 to coil assemblies on the headset 403. The external medical device 402 and the coil assemblies may be configured to wireless interface (e.g., inductive charging and/or communication) with implantable devices 400. The coil assemblies may be referred to as patient-applied-parts. The illustrated system 417 may further include at least one sensor 418, such as a temperature sensor in or near a coil assembly. The external medical device 402 may include a power source 419 such as a battery or rechargeable battery, a control circuit 420 for interfacing with the coil assemblies using power from the power source, and an electrical port 421 for connecting to a connector (e.g., plug) on a proximal end of the headset cable 407. The cable 407 may include two sizes of wires. A smaller gauge of wires 422 may be used for the transmission of lower power signals, and a larger gauge of wires 423 may be used for higher power signals. Sensor signals, such as temperature signals, may be transmitted using lower power signals on the small gauge of wires. The coil assemblies may be energized for charging, programmimg, and communicating using the large gauge of wires.

FIG. 5 illustrates, by way of example and not limitation, a plug or connector 524 on a proximal end of the cable 507 for connection to the port of the external medical device. The plug 524 may include contacts conductively attached to the wires and to shielding in the cable. For example, FIG. 5 illustrates the plug as having a socket for a 9-pin connector. FIG. 6 illustrates, by way of example and not limitation, a port 621 in the external medical device 602 for connecting to the plug 524 on the proximal end of the cable 507 (see FIG. 5). The plug and the port are configured to electrically connect cable wires and cable shielding to the external medical device.

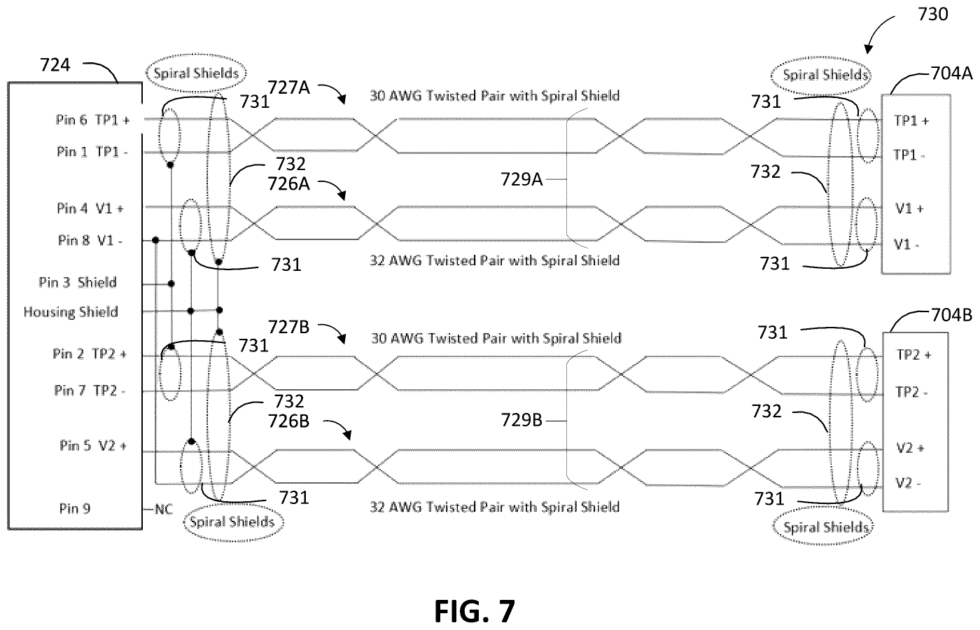

FIG. 7 illustrates, by way of example and not limitation, a schematic diagram for the cable. The illustrated cable includes four twisted wire pairs, including two smaller gauge (e.g., 32 AWG) twisted wire pairs 726A and 726B and two larger gauge (e.g., 30 AWG) twisted wire pairs 727A and 727B. The proximal end for each of the twisted wire pairs 726A, 726B, 727A, 727B is connected to the connector or plug 724. The four twisted wire pairs 726A, 726B, 727A, 727B are grouped into a first set 729A and a second set 729B, where the first set 729A includes the wire pairs 726A and 727A and the second set 729B includes the wire pairs 726B and 727B. The first set 729A of wire pairs 726A and 727A may be hardwired to a first external coil assembly 704A (e.g., via a terminal block), and second set 729B of wire pairs 726B and 727B may be hardwired to a second external coil assembly 704B (e.g., via a terminal block) at their distal ends 730. Alternatively, the connections to the external coil assemblies 704A and 704B may be a removable connection such as a pin and socket connector.

Each wire in each of the twisted wire pairs 726A, 726B, 727A, 727B may be coated with Perfluoroalkoxy (PFA) (e.g., TEFLON). Each of the twisted pairs may be wrapped with a conductive shield (e.g., a “twisted-pair shield”). Each of first and second sets 729A and 729B may include a shield (referred to herein as a “conductive jacket shield”) 732 as is may be formed within an insulator jacket of the cable. All the shields 731, 732 may be terminated together at the connector to the external medical device and may connect to the system ground via the external medical device so that the system is less affected by any common mode interference from the signals and shields.

Each twisted pair has a twisted pair shield (e.g. spiral PTFE shields). There is an overall spiral shield within each cable that wraps around both twisted pairs, Each shield is wrapped with PTFE (e.g., TEFLON brand) tape. The two cables are fused together and can be pulled apart along the fuse, A yoke (e.g., and O-ring or similar feature) may be used to prevent further cable separation.

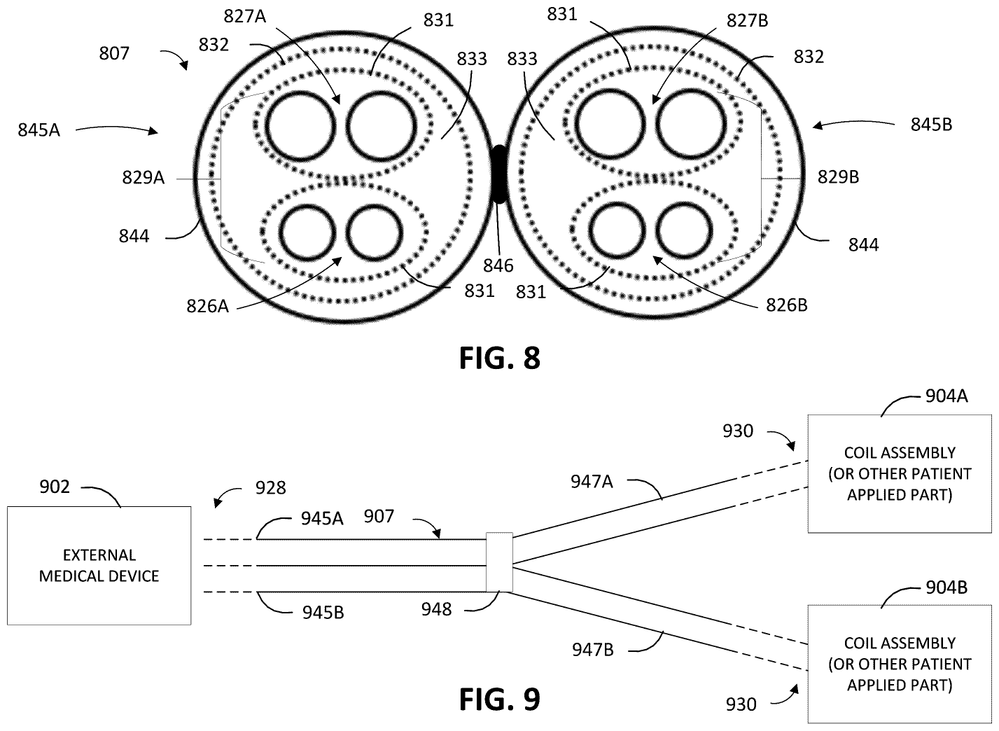

FIG. 8 illustrates, by way of example and not limitation, a cross-section of the cable 807 with a zip cord construction. The illustrated cable 807 includes four twisted wire pairs, including two smaller gauge (e.g., 32 AWG) twisted wire pairs 826A and 826B and two larger gauge (e.g., 30 AWG) twisted wire pairs 827A and 827B. Each of the twisted pairs may be wrapped with a shield (e.g., a “twisted-pair shield”) 831.

The four twisted wire pairs 826A, 826B, 827A, 827B are grouped into a first set 829A and a second set 829B, where the first set 829A includes the wire pairs 826A and 827A and the second set 829B includes the wire pairs 826B and 827B. An insulator jacket 833 may be formed around each of the first set 829A and second set 829B, and a shield (referred to herein as a “conductive jacket shield”) 832 may surround each insulator jacket 833. A cable coating (e.g., PTFE) 844 may be over the conductive jacket shield. The insulating jacket materials on the zip cord cable may be polyvinyl chloride (PVC), such as white PVC. The shields may be tinned copper spiral wound shields. Spiral wound shields provide 360 degrees of shielding and is a less expensive shielding method than other options such as an extruded contiguous shield. Individual conductors are insulated with PFA and each shield for the twisted pairs and for each side of the zip cord is insulated with a PTFE wrap to electrically insulate the shields from one another over the length of the cable. The PFA and PTFE insulation also reduces friction between individual twisted pairs within each side of the zip cord which can reduce noise between the signals. The high power, heavier gauge twisted pair wires are impedance controlled to 75 ohms to provide a higher efficiency transfer of power. The cable may be formed as two side-by-side insulated cable segments 845A and 845B fused together at a “fused region” 846. These cable segments 845A and 845B may be pulled apart to form corresponding distal segments. Two polyester filler cords may be placed in each side of the zip cord to internally support the round shape of each side of the zip cord.

FIG. 9 illustrates a block diagram of the cable 907 with the zip cord construction with a proximal end 928 for connecting to the external medical device 901, where the distal end 930 of the cable is separated, such that the cable segments 945A and 945B are separated from each other to form two separate distal segments 947A and 947B for connecting to the two coil assemblies 904A and 904B. A yoke 948, such as an O-ring, may be used to limit how far cable segments 945A and 945B can be separated so that the proximal end 928 continues to have two cable segments 945A and 945B fused together to form one proximal cable where it connects to the external medical device 902,

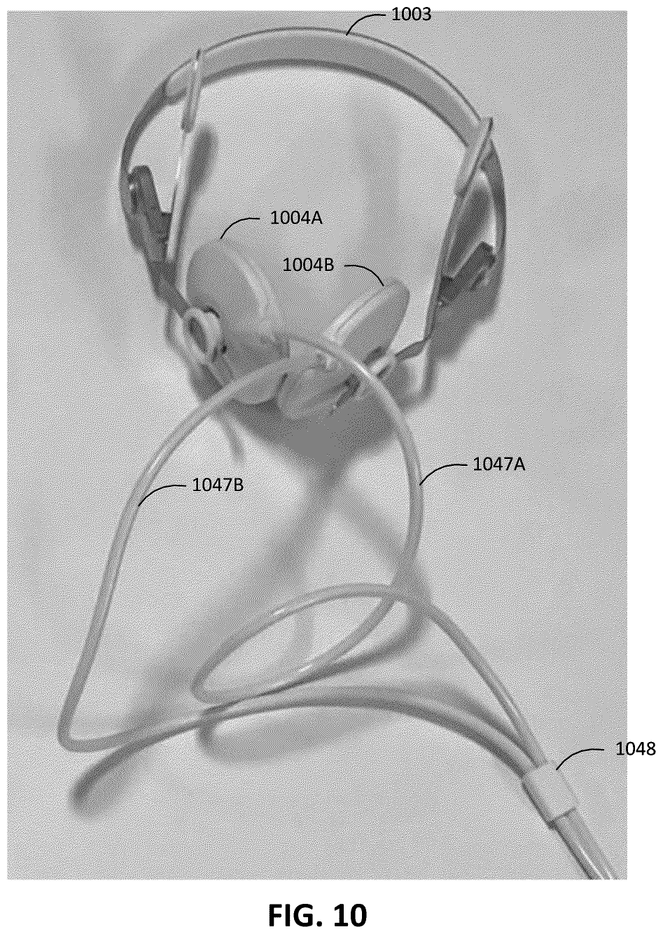

FIG. 10 illustrates a headset with wires within the distal ends of the distal segments 1047A and 1047B hardwired to coil assemblies 1004A and 1004B on the headset 1003. FIG. 10 also illustrates an example of a yoke 1048. FIG. 10 illustrates an example of a headset cable. An operating room (OR) cable may be attached to coils assemblies, but not attached to the headset. The OR cable may have a longer cable to conned from outside; to inside of the sterile field in the operating room such that each coil assembly can be positioned over each implant while placed in a sterile sleeve for use in the sterile field.

A bilateral implant (e.g., two patient-applied parts) was described above. However, the cable construction may be modified to handle more than two patient-applied parts, by including a cable segment for every separately controlled patient-applied part. The shields may include spiral wound conductive shields, or other shield types such as solid conductive shields, mesh shields, and the like. The cable construction describes two sets of conductors to each patient-applied part. However, the cable may be constructed to provide a single set or multiple sets of conductors to each patient-applied part. These sets of conductors could be all higher-power-signal conductors, all lower power signal conductors, or any combination of both. Higher-power-signal conductors have a higher current capacity than the lower power-signal-conductors.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A medical system, comprising:
an external medical device;
at least two patient-applied parts configured to physically contact a patient during normal use;
a furcated cable having one proximal end configured to be connected to the external medical device and having at least two distal ends, including a first distal end and a second distal end, configured to be connected to the at least two patient-applied parts, the cable including:
first and second types of twisted wire pairs, the second type of twisted wire pairs including wires that have a lower gauge and higher current capacity than the first type of twisted wire pairs,
at least four sets of twisted wire pairs, including at least two sets of twisted wire pairs of the first type and at least two sets of twisted wire pairs of the second type,
a first one of the at least two sets of twisted wire pairs of the first type and a first one of the at least two sets of twisted wire pairs of the second type extending from the proximal end to the first distal end; and
a second one of the at least two sets of twisted wire pairs of the first type and a second one of the at least two sets of twisted wire pairs of the second type extending from the proximal end to the second distal end.

2. The medical system of claim 1, wherein the external medical device includes an electrical port, and the one proximal end of the cable includes one connector plug for electrically connecting the at least four twisted wire pairs in the cable to the electrical port.

3. The medical system of claim 1, wherein
the first one of the at least two sets of twisted wire pairs of the first type and the first one of the at least two sets of twisted wire pairs of the second type are hardwired at the first distal end to a first one of the at least two patient-applied parts; and
the second one of the at least two sets of twisted wire pairs of the first type and the second one of the at least two sets of twisted wire pairs of the second type are hardwired at the second distal end to a second one of the at least two patient-applied parts.

4. The medical system of claim 1, wherein each of the at least four sets of twisted pairs has a conductive shield.

5. The medical system of claim 4, wherein the external medical device includes a reference or ground node, and the conductive shields are terminated together and electrically connected to the reference or ground node in the external medical device.

6. The medical system of claim 4, wherein the conductive shield includes a tinned copper spiral wound shield.

7. The medical system of claim 6, wherein each wire within the at least four sets of twisted wire pairs is insulated with Perfluoroalkoxy (PFA) and each of the conductive shields are insulated with polytetrafluoroethylene (PTFE) tape.

8. The medical system of claim 1, wherein:
the furcated cable is a zip cord style cable configured to be pulled apart into at least a first distal segment near the first distal end and a second distal segment near the second distal end;
the first distal segment includes distal portions of the first one of the at least two sets of twisted wire pairs of the first type and the first one of the at least two sets of twisted wire pairs of the second type; and
the second distal segment includes distal portions of the second one of the at least two sets of twisted wire pairs of the first type and the second one of the at least two sets of twisted wire pairs of the second type.

9. The medical system of claim 8, wherein the cable includes a yoke to keep the zip cord style cable from separating into the first and second distal segments past the yoke.

10. The medical system of claim 8, wherein the zip cord style cable includes:
a first insulator jacket around the first one of the at least two sets of twisted wire pairs of the first type and the first one of the at least two sets of twisted wire pairs of the second type; and
a second insulator jacket around the second one of the at least two sets of twisted wire pairs of the first type and the second one of the at least two sets of twisted wire pairs of the second type, the first insulator jacket being adjacent and fused to the second insulator jacket, wherein the first and second insulator jackets are configured to be separated along the fuse to provide the first distal segment using the first insulator jacket and provide the second distal segment using the second insulator jacket.

11. The medical system of claim 10, wherein the first and second insulator jackets include polyvinyl chloride (PVC).

12. The medical system of claim 10, further comprising:
a first conductive jacket shield around the first one of the at least two sets of twisted wire pairs of the first type and the first one of the at least two sets of twisted wire pairs of the second type, the first insulator jacket coating the first conductive jacket shield; and
a second conductive jacket shield around the second one of the at least two sets of twisted wire pairs of the first type and the second one of the at least two sets of twisted wire pairs of the second type, the second insulator jacket coating the second conductive jacket shield.

13. The medical system of claim 1, wherein the patient-applied part includes:
a coil assembly configured for use to be placed on the patient and used to charge, program, or communicate with an implantable device; or
at least one electrode configured to contact the patient, wherein the at least one electrode is used to deliver an electrical therapy to the patient or to sense an electrical signal from the patient.

14. The medical system of claim 1, wherein the first type and the second type of twisted wire pairs include 32 AWG and 30 AWG wires, respectively.

15. The medical system of claim 1, wherein the medical system is configured to diagnose, treat or monitor a patient or to compensate or alleviate a disease, an injury or a disability experienced by the patient by the at least two patient-applied parts physically contacting the patient during normal use.

16. A medical system for communicating with at least two implanted devices, comprising:
at least two coil assemblies corresponding to the at least two implanted medical devices, wherein each of the at least two coil assemblies is configured for use to charge, program, and/or communicate with a corresponding one of the at least two implanted devices;
an external medical device; and
a cable for providing an electrical connection between the external medical device and the at least two coil assemblies,
wherein the cable includes first and second types of twisted wire pairs, the second type of twisted wire pairs being configured to conduct higher power signals than the first type of twisted wire pairs,
wherein the cable includes at least four sets of twisted wire pairs, including at least two sets of twisted wire pairs of the first type and at least two sets of twisted wire pairs of the second type, and
wherein each of the at least two coil assemblies is connected to one of the at least two sets of twisted wire pairs of the first type and is further connected to one of the at least two sets of twisted wire pairs of the second type.

17. The medical system of claim 16, wherein the first type and the second type of twisted wire pairs include 32 AWG and 30 AWG wires, respectively.

18. The medical system of claim 16, further comprising a headset, the at least two coil assemblies including a first coil assembly attached to the headset and a second coil assembly attached to the headset.

19. The medical system of claim 16, wherein the second type of twisted wire pairs includes larger gauge wires than the first type of twisted wire pairs, the second type being used to provide at least one of: power or recharging to the implanted device, communication with the implanted device, or programming signals to program the implanted devices.

20. The medical system of claim 16, wherein the first type of twisted wire pairs includes smaller gauge wires than the second type of twisted wire pairs, the first type being used to provide sensor signals.

21. The medical system of claim 20, wherein the sensor signals include a temperature sensor signal.

22. A medical system, comprising:
a headset including a first coil assembly and a second coil assembly, wherein the headset is configured to be worn on the head to place the first coil assembly against the head over a first head-located implant and place the second coil assembly against the head over a second head-located implant;
an external medical device including an electrical port;
a cable for providing an electrical connection between the external medical device and the first and second coil assemblies, wherein the external medical device is configured to charge, program, and/or communicate with the first and second head-located implants using the first and second coils assemblies, respectively;
the cable including a proximal end and a bifurcated distal end forming a first distal segment with a first distal end and forming a second distal segment with a second distal end, the proximal end including a single connector plug for connecting to the electrical port;
the cable including four twisted wire pairs, wherein the single connector plug is configured to electrically connect the four twisted wire pairs to the external medical device via the electrical port, the four twisted wire pairs including first and second twisted wire pairs of a first gauge and first and second twisted wire pairs of a second gauge larger than the first gauge, each wire within the at least four twisted wire pairs is coated with Perfluoroalkoxy(PFA) and a first conductive shield around the first twisted wire pair and a second conductive shield around the second twisted wire pair are insulated with polytetrafluoroethylene PTFE tape; and
the cable including:
a first insulator jacket around the first conductive shield around the first twisted pair of the first gauge and the first twisted pair of the second gauge; and
a second insulator jacket around the second conductive shield around the second twisted pair of the first gauge and the second twisted pair of the second gauge, the first insulator jacket being fused with the second insulator jacket such that the cable is a zip cord style cable capable of having the first and second insulator jackets separated to form a first distal segment and a second distal segment,
wherein the first twisted pair of the first gauge and the first twisted pair of the second gauge in the first distal segment are hardwired to the first coil assembly, and the second twisted pair of the first gauge and the second twisted pair of the second gauge being hardwired to the second coil assembly.

23. The medical system of claim 22, wherein the zip cord style cable includes a yoke configured to limit separation of the first and second insulator jackets into the first and second distal segments, respectively.

24. The medical system of claim 22, wherein each of the at least four sets of twisted pairs has a conductive shield.

25. The medical system of claim 24, wherein the external medical device includes a reference or ground node, and the conductive shields are terminated together and electrically connected to the reference or ground node in the external medical device.

26. The medical system of claim 24, further comprising:
a first conductive jacket shield corresponding to the first insulator jacket, the first conductive jacket shield being wrapped around the shield of the first twisted pair of the first gauge and the first twisted pair of the second gauge; and
a second conductive jacket shield corresponding to the second insulator jacket, the second conductive jacket shield being wrapped around the shield of the second twisted pair of the first gauge and the second twisted pair of the second gauge.

27. The medical system of claim 26, wherein the first and second conductive jacket shields are insulated with polytetrafluoroethylene (PTFE) tape.

28. The medical system of claim 22, wherein the first gauge is 32 AWG and the second gauge is 30 AWG.

29. The medical system of claim 22, wherein the headset further includes a sensor, and the external medical device is configured to use the first and second twisted pairs of the first gauge to communicate with the sensor, and is configured to use the first and second twisted pairs of the second gauge to charge, program, and communicate with the head-located implants.

30. The medical system of claim 22, wherein the cable further includes shielding, the electrical port includes a multi-pin connector, and the connector plug is configured to be inserted into the multi-pin connector, wherein a plurality of pins is used to connect to the four twisted wire pairs, and at least one pin is used to connect shielding to a reference node or ground node within the external medical device.
